(19) United States
(12) Patent Application Publication
(10) Pub. No.: US 2015/0147406 A1
(43) Pub. Date: May 28, 2015

(54) Intraocular Formulation

(71) Applicant: Allergan, Inc., Irvine, CA (US)

(72) Inventors: Michael R. Robinson, Irvine, CA (US); Wendy M. Blanda, Tustin, CA (US); Hui Liu, Irvine, CA (US); Scott M. Whitcup, Laguna Hills, CA (US); Patrick M. Hughes, Aliso Viejo, CA (US)

(21) Appl. No.: 14/298,418

(22) Filed: Jun. 6, 2014

Related U.S. Application Data
(63) Continuation of application No. 12/626,408, filed on Nov. 25, 2009, now abandoned.
Application No. 12/626,408 is a continuation of application No. 11/952,938, filed on Dec. 7, 2007, now abandoned.
Application No. 11/952,938 is a continuation-in-part of application No. 11/116,698, filed on Apr. 27, 2005, now abandoned.
Application No. 11/116,698 is a continuation-in-part of application No. 10/966,764, filed on Oct. 14, 2004, now abandoned.
(60) Provisional application No. 60/567,423, filed on Apr. 30, 2004.
(60) Provisional application No. 60/519,237, filed on Nov. 12, 2003.
(60) Provisional application No. 60/530,062, filed on Dec. 16, 2003.

(51) Int. Cl. A61K 9/16; A61K 9/00; A61K 47/36; A61K 31/573; C07K 16/22

(52) U.S. Cl. CPC A61K 9/1647; A61K 31/573; C07K 16/22; A61K 47/36; A61K 9/0048; C07K 2317/76
USPC 424/501; 514/180; 424/145.1

(57) ABSTRACT

Biodegradable therapeutic agent incorporating microspheres formulated in a high viscosity carrier suitable for intraocular administration to treat an ocular condition. The formulation can also be used to treat non-ocular conditions such as articular pathologies

### CROSS REFERENCE TO RELATED APPLICATIONS

This application is a continuation of U.S. application Ser. No. 12/626,408 filed Nov. 25, 2009; a continuation of U.S. application Ser. No. 11/952,938 filed Dec. 7, 2007; a continuation-in-part of U.S. application Ser. No. 11/116,698 filed Apr. 27, 2005 which claims the benefit of U.S. Application No. 60/567,423, filed Apr. 30, 2004; and a continuation-in-part of U.S. application Ser. No. 10/966,764 filed Oct. 14, 2004 which claims the benefit of U.S. Provisional Patent Application Ser. No. 60/519,237, filed Nov. 12, 2003 and U.S. Provisional Patent Application Ser. No. 60/530,062, filed Dec. 16, 2003. Each of these applications is fully incorporated by reference herein.

### BACKGROUND

The present invention relates to intraocular formulations for treating ocular conditions. In particular the present invention relates to intraocular formulations comprising a plurality of therapeutic agent incorporating, biodegradable microspheres formulated with a high viscosity carrier to treat a variety of ocular conditions.

An ocular condition can include a disease, aliment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball. A front of the eye ocular condition is a disease, ailment or condition which affects or which involves an ocular region or site, such as a periocular muscle, an eye lid or an eye ball tissue or fluid which is located anterior to the posterior wall of the lens capsule or ciliary muscles. Thus, a front of the eye ocular condition primarily affects or involves, the conjunctiva, the cornea, the conjunctiva, the anterior chamber, the iris, the posterior chamber (behind the iris but in front of the posterior wall of the lens capsule), the lens and the lens capsule as well as blood vessels, lymphatics and nerves which vascularize, maintain or innervate an anterior ocular region or site.

A front of the eye ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can be considered to be a front of the eye ocular condition because a clinical goal of glaucoma treatment can be to reduce a hypertension of aqueous fluid in the anterior chamber of the eye (i.e. reduce intraocular pressure).

A posterior (back of the eye) ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site.

Thus, a posterior ocular condition can include a disease, ailment or condition, such as for example, macular degeneration (such as non-exudative age related macular degeneration and exudative age related macular degeneration); choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can also be considered a posterior ocular condition because a therapeutic goal of glaucoma treatment is to prevent the loss of or reduce the occurrence of loss of vision due to damage to or loss of retinal cells or optic nerve cells (i.e. neuroprotection).

The exterior surface of the normal globe mammalian eye has a layer of tissue known as conjunctival epithelium, under which is a layer of tissue called Tenon's fascia (also called conjunctival stroma). The extent of the Tenon's fascia extending backwards across the globe forms a fascial sheath known as Tenon's capsule. Under Tenon's fascia is the episclera. Collectively, the conjunctival epithelium and the Tenon's fascia is referred to as the conjunctiva. As noted, under Tenon's fascia is the episclera, underneath which lies the sclera, followed by the choroid. Most of the lymphatic vessels and their associated drainage system, which is very efficient at removing therapeutic agents placed in their vicinity, is present in the conjunctiva of the eye.

It is known to administer a drug depot to the posterior (i.e. near the macula) sub-Tenon space. See eg column 4 of U.S. Pat. No. 6,413,245. Additionally, it is known to administer a polylactic implant to the sub-tenon space or to a suprachoroidal location. See eg published U.S. Pat. No. 5,264,188 and published U.S. patent application 20050244463

An intraocular drug delivery system can be made of a biodegradable polymeric such as a poly(lactide) (PLA) polymers, poly(lactide-co-glycolide) (PLGA) polymers, as well as copolymers of PLA and PLGA polymers. PLA and PLGA polymers degrade by hydrolysis, and the degradation products, lactic acid and glycolic acid, are metabolized into carbon dioxide and water.

Drug delivery systems have been formulated with various active agents. For example, it is known to make 2-methoxyestradiol poly lactic acid polymer implants (as rods and wafers), intended for intraocular use, by a melt extrusion method.

See eg published U.S. patent application 20050244471. Additionally, it is known to make brimonidine poly lactic acid polymer implants and microspheres intended for intraocular use. See eg published U.S. patent applications 20050244463 and 20050244506, and U.S. patent application Ser. No. 11/395,019. Furthermore, it is known to make bimatoprost containing polylactic acid polymer implants and microspheres intended for intraocular use. See eg published U.S. patent applications 2005 0244464 and 2006 0182781, and U.S. patent application Ser. Nos. 11/303,462, and; 11/371,118.

Intraocular drug delivery systems which are sutured or fixed in place are known. Suturing or other fixation means requires sensitive ocular tissues to be in contact with aspects of a drug delivery system which are not required in order to contain a therapeutic agent within or on the drug delivery system or to permit the therapeutic agent to be released in vivo. As such suturing or eye fixation means a merely peripheral or ancillary value and their use can increase healing time, patient discomfort and the risk of infection or other complications.

An intraocular drug delivery system can be in the form of a biodegradable implant. The implant is prepared to include a therapeutic agent which is released upon intraocular placement of the implant. Being biodegradable the implant need not be removed once it has been implanted and has released the therapeutic agent incorporated therein. An implant can be a disc, fiber or rod with relatively large dimensions (i.e. about 0.5 mm by 2-6 mm). Because of their size such implant can require an incision, result in patient discomfort, obscure vision, and/or cause hyperemia. Alternately the implant can comprise a population of individually much smaller biodegradable microspheres which can be more likely to completely biodegrade and be injectable through a larger gauge (smaller needle diameter) syringe, as compared to larger implant.

U.S. patent applications which disclose intraocular use of microspheres and/or use of a therapeutic agent formulated with a hyaluronic acid include application Ser. No. 11/070,158, filed Mar. 1, 2005, application Ser. No. 11/118,519, filed Apr. 29, 2005, application Ser. No. 11/368,845, filed Mar. 6, 2006, application Ser. No. 11/371,118, filed Mar. 8, 2006, application Ser. No. 11/119,463, filed Apr. 29, 2005, application Ser. No. 11/565,917, filed Dec. 1, 2006, application Ser. No. 11/565,917, application Ser. No. 10/966,764, filed Oct. 14, 2004, application Ser. No. 11/091,977, Mar. 28, 2005, application Ser. No. 11/354,415, Feb. 14, 2006, application Ser. No. 11/741,366, Apr. 27, 2007, application Ser. No. 11/828,561, Jul. 26, 2007, application Ser. No. 11/039,192, filed Jan. 19, 2005, application Ser. No. 11/116,698, filed Apr. 27, 2005, application Ser. No. 11/695,527, filed Apr. 2, 2007, and application Ser. No. 11/742,350, filed Apr. 30, 2007.

Artecoll is manufactured by Rofil Medical International. It is marketed in Canada. Artecoll is not FDA approved in the United States. Artecoll consists of polymethylmethacrylate microspheres suspended in bovine collagen. The collagen serves as a vehicle for injection and is eventually degraded, leaving behind permanent implantation of the non-biodegradable beads. The mixture is injected at the dermal-subdermal junction to treat deeper rhytids and scars. Patients must be tested for allergy to bovine collagen prior to administration.

Radiesse is manufactured by Bioform. Radiesse is composed of microspheres of calcium hydroxyl appetite suspended in an aqueous gel carrier. These biodegradable microspheres serve as a lattice upon which the body forms a scaffold for tissue infiltration. The spheres degrade slowly over years for a longer-lasting, semi-permanent effect. Radiesse is approved by the FDA for such indications as bladder neck augmentation for urinary incontinence, vocal cord augmentation for paresis, and periodontal defects. Its use as a soft tissue filler is off-label. The most commonly treated areas are the nasolabial folds and marionette lines.

Reviderm intra is manufactured by Roth Medical International. This product is not FDA approved in the United States. Reviderm intra consists of 40- to 60-micron dextran beads suspended in hylan gel of nonanimal origin. The proposed mechanism of action is an initial macrophage response followed by fibroblast proliferation and new collagen formation. Intradermal injection is used to treat rhytids and cutaneous defects (eg, atrophic scars), and for lip augmentation.

Microspheres, commonly made with PLGA (poly(lactic-co-glycolic) acid) and other bioerodible polymers, have been used for intravitreal drug delivery for retinal diseases. See eg Giordano G. et al, Sustained delivery of retinoic acid from microspheres of biodegradable polymer in PVR, Invest Ophthalmol Vis Sci 1993; 34:2743-51, and; Herrero-Vanrell R, et al., Biodegradable microspheres for vitreoretinal drug delivery, Adv Drug Deliv Rev 2001; 52:5-16. Unfortunately, following intravitreal injection, microspheres can lead to an acute inflammatory response in the vitreous and retina. Bourges J. et al., Ocular drug delivery targeting the retina and retinal pigment epithelium using polylactide nanoparticles, Invest Ophthalmol Vis Sci 2003; 44:3562-9.

A problem which can occur upon in vivo administration of therapeutic agent incorporating microspheres is inflammation. A significant factor associated with the inflammatory reaction after microsphere injection is the diffuse distribution of particles following injection which stimulates macrophage activity. Phagocytosis leads to cytokine release and both neutrophils and additional macrophages are recruited. The enormous numbers of microspheres can be lethal to macrophages and neutrophils, causing these cells to die and release lysosomal contents, oxidative enzymes, and more proinflammatory cytokines. This results in an acceleration of the inflammatory reaction. This toxic inflammatory response to particles is observed clinically with crystal synovitis, observed following injection of corticosteroid particles in the joint. McCarty D. et al., Inflammatory Reaction after Intrasynovial Injection of Microcrystalline Adrenocorticosteroid Esters, Arthritis Rheum 1964; 7:359-67. This inflammatory response can also occur with indigestible crystals or particles that are not pharmaceutical in nature, the classic example being the disease gout. Here, patients develop a chronic synovitis and arthritis from urate crystals damaging macrophages following phagocytosis. McCarty D., Crystal-induced inflammation; syndromes of gout and pseudogout, Geriatrics 1963; 18:467-78. Inflammation from particle exposure in the vitreous cavity has been recognized following intravitreal injections of Kenalog-40. Patients can present typically the following day with vision loss with a clinical condition known as sterile endophthalmitis. See e.g. Wang L. et al., Sterile endophthalmitis following intravitreal injection of triamcinolone acetonide, Ocul Immunol Inflamm 2005; 13:295-300, and; Taban M. et al., Sterile endophthalmitis after intravitreal triamcinolone: a possible association with uveitis, Am J Ophthalmol 2007; 144:50-54. A similar inflammatory response to corticosteroid particles can occur following injections into the sub-Tenon's space. Giangiacomo J. et al., Histopathology of triamcinolone in the subconjunctiva, Ophthalmology 1987; 94:149-53.

Thus significant problems can occur upon intraocular injection of known formulations of microspheres including rapid dispersal of the microspheres, which can obscure the patient's vision, rapid removal of the microspheres from the injection site (as by the lymphatic drainage system or by phagoctyosis) which reduces drug effect at the locus of the target site injection, and immunogenicity upon recognition of the microspheres by macrophages and other elements of the immune system. Thus, there is a need for an intraocular formulation for the treatment of an ocular condition which addresses these problems.

### SUMMARY

The present invention meets these and other needs and provides an intraocular formulation which is a microsphere based drug delivery system for the treatment of an ocular condition which can provide sustained release of a therapeutically effective amount of a therapeutic agent without rapid dispersal of the microspheres and hence little or no obscuration of the patient's vision, without rapid removal of the microspheres from the injection site (so that drug delivery at the locus of the target site injection in increased), and with a reduced immunogenicity upon injection of the therapeutic agent incorporating microspheres.

### DEFINITIONS

The terms below are defined to have the following meanings:

“About” means approximately or nearly and in the context of a numerical value or range set forth herein means ±10% of the numerical value or range recited or claimed.

“Active agent”, “drug” and “therapeutic agent” are used interchangeably herein and refer to any substance used to treat an ocular condition or a non-ocular condition, such as an articular pathology.

“Anterior intraocular location” with regard to a site of administration of a drug delivery system for treatment of an ocular hypertensive condition means a sub-Tenon, suprachoroidal, intrascleral, episcleral, and the like intraocular location which is located no more than about 10 mm (preferably no more than about 8 mm) along the curvature of the surface of the eye from the corneal limbus.

“Bioerodible polymer” means a polymer which degrades in viva Drug delivery systems containing bioerodible polymers can have a triphasic pattern of drug release: an initial burst from surface bound drug; the second phase from diffusional release, and; release due to degradation of the polymer matrix. Thus, erosion of the polymer over time is required to release all of the active agent. Hydrogels such as methylcellulose can release drug through polymer swelling. The words “bioerodible” and “biodegradable” are synonymous and are used interchangeably herein.

“Cumulative release profile” means to the cumulative total percent of an active agent released from an implant into an ocular region or site in vivo over time or into a specific release medium in vitro over time.

“Drug delivery system” means a physical device from which a therapeutic amount of a therapeutic agent can be released upon in vivo administration of the drug delivery system. The drug delivery system can be an implant (which can be configured for example as a rod, cylinder, filament, fiber, disc or wafer) or a population of microspheres. “Implant” includes a plurality of microspheres.

“Glaucoma” means primary, secondary and/or congenital glaucoma. Primary glaucoma can include open angle and closed angle glaucoma. Secondary glaucoma can occur as a complication of a variety of other conditions, such as injury, inflammation, vascular disease and diabetes.

“Inflammation-mediated” in relation to an ocular condition means any condition of the eye which can benefit from treatment with an anti-inflammatory agent, and is meant to include, but is not limited to, uveitis, macular edema, acute macular degeneration, retinal detachment, ocular tumors, fungal or viral infections, multifocal choroiditis, diabetic uveitis, proliferative vitreoretinopathy (PVR), sympathetic opthalmia, Vogt Koyanagi-Harada (VKH) syndrome, histoplasmosis, and uveal diffusion.

“Injury” or “damage” are interchangeable and refer to the cellular and morphological manifestations and symptoms resulting from an inflammatory-mediated condition, such as, for example, inflammation.

“Intraocular” means within or under an ocular tissue. An Intraocular administration of a drug delivery system includes administration of the drug delivery system to a sub-Tenon, subconjunctival, suprachoroidal, intravitreal and like location. An intraocular administration of a drug delivery system excludes administration of the drug delivery system to a topical, systemic, intramuscular, subcutaneous, intraperitoneal, and the like location.

“Ocular condition” means a disease, aliment or condition which affects or involves the eye or one or the parts or regions of the eye, such as a retinal disease. The eye includes the eyeball and the tissues and fluids which constitute the eyeball, the periocular muscles (such as the oblique and rectus muscles) and the portion of the optic nerve which is within or adjacent to the eyeball.

“Plurality” means two or more.

“Posterior ocular condition” means a disease, ailment or condition which affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerve which vascularize or innervate a posterior ocular region or site.

“Steroidal anti-inflammatory agent” and “glucocorticoid” are used interchangeably herein, and are meant to include steroidal agents, compounds or drugs which reduce inflammation when administered at a therapeutically effective level.

“Substantially” in relation to the release profile or the release characteristic of an active agent from a bioerodible implant as in the phrase “substantially continuous rate” of the active agent release rate from the implant means, that the rate of release (i.e. amount of active agent released/unit of time) does not vary by more than 100%, and preferably does not vary by more than 50%, over the period of time selected (i.e. a number of days). “Substantially” in relation to the blending, mixing or dispersing of an active agent in a polymer, as in the phrase “substantially homogenously dispersed” means that there are no or essentially no particles (i.e. aggregations) of active agent in such a homogenous dispersal.

“Suitable for insertion (or implantation) in (or into) an ocular region or site” with regard to an implant, means an implant which has a size (dimensions) such that it can be inserted or implanted without causing excessive tissue damage and without unduly physically interfering with the existing vision of the patient into which the implant is implanted or inserted.

“Sustained” as in “sustained period” or “sustained release” means for a period of time greater than thirty days, preferably for at least 20 days (i.e. for a period of time from 20 days to 365 days), and most preferably for at least 30 days. A sustained release can persist for a year or more.

“Therapeutic levels” or “therapeutic amount” means an amount or a concentration of an active agent that has been locally delivered to an ocular region that is appropriate to safely treat an ocular condition so as to reduce or prevent a symptom of an ocular condition.

Our invention encompasses a drug delivery system for treating an ocular condition, the drug delivery system can comprise: (a) at least one bioerodible implant suitable for insertion into an ocular region or site, the bioerodible implant comprising; (i) an active agent, and; (ii) a bioerodible polymer, wherein the bioerodible implant can release a therapeutic level of the active agent into the ocular region or site for a period time between about 30 days and about 1 year. Preferably, the bioerodible implant can release the therapeutic level of the active agent into the ocular region or site at a substantially continuous rate in vivo. More preferably, the bioerodible implant can release a therapeutic level of the active agent into the ocular region or site at a substantially continuous rate upon implantation in the vitreous for a period time between about 50 days and about 1 year. The active agent can be an anti-inflammatory agent. The bioerodible polymer can be a PLGA co-polymer.

The bioerodible implant can have a weight between about 1 μg and about 1 g and no dimension less than about 0.1 mm and no dimension greater than about 20 mm. Preferably, the drug delivery system comprises a plurality of microspheres each with a size (diameter) of from about 50 nm to about 200 microns.

A drug delivery system of claim within the scope of our invention can comprise a plurality of bioerodible microspheres. The active agent can be substantially homogenously dispersed within the bioerodible polymer or the active agent can be associated with the bioerodible polymer in the form of particles of active agent and bioerodible polymer.

In a preferred embodiment the drug delivery system can comprise: (a) a portion of the active agent substantially homogenously dispersed within a portion of the bioerodible polymer, and; (b) a portion of the same or of a different active agent associated with a portion of same or of a different bioerodible polymer in the form of particles of active agent and the bioerodible polymer.

In a further embodiment the drug delivery system can comprise: (a) a bioerodible implant suitable for insertion into an ocular region or site, the bioerodible implant comprising; (i) an active agent, and; (ii) a bioerodible polymer, wherein the bioerodible implant can release a therapeutic level of the active agent upon insertion into a posterior ocular region or site for a period time of at least about 40 days.

Our invention is a biocompatible, injectable intraocular drug delivery system comprising: (a) a plurality of biodegradable microspheres, (b) a therapeutic agent incorporated by the microspheres, and (c) a viscous carrier for the microspheres, the viscous carrier having a viscosity of at least about 10 cps at a shear rate of 0.1/second at 25° C., thereby forming a biocompatible, injectable intraocular drug delivery system. The drug delivery system can further comprising an aqueous vehicle for the microspheres.

The therapeutic agent in the drug delivery system can have a solubility in water at 25° C. of between about 0.1 μg/ml and about 1 gm/ml and the drug delivery can be injectable into an intraocular location through a 25 to 32 gauge syringe needle.

With our drug delivery system the viscous carrier can have a viscosity at 25° C. of between about 140,000 cps and about 500,000 cps at a shear rate of 0.1/second and the microspheres can be substantially uniformly suspended in the viscous carrier composition. The present drug delivery system can include a non-cross linked hyaluronic acid, a cross linked hyaluronic acid and combinations thereof. For properties of cross linked hyaluronic acids see eg U.S. Pat. No. 6,831,172.

The therapeutic agent can be a corticosteroid and the viscous carrier can be a hyaluronic acid, such as a non-cross-linked or a cross-linked hyaluronic acid. The cross-linked polymeric hyaluronic acid can have a molecular weight of about 1 million Daltons.

With our drug delivery system the microspheres can comprise a poly lactide, co-glyclolide (PLGA) polymer and have an average diameter between about 1 microns and about 100 microns.

A detailed embodiment of our invention can be a biocompatible, injectable intraocular drug delivery system comprising:
(a) a plurality of biodegradable microspheres, wherein the microspheres comprise a polylactide, co-glyclolide (PLGA) polymer and the microspheres have an average diameter between about 1 microns and about 100 microns,
(b) a corticosteroid. incorporated by the microspheres, wherein the corticosteroid has a solubility in water at 25° C. of between about 0.1 mg/ml and about 1 gm/ml,
(c) an aqueous vehicle for the microspheres, and
(d) a hyaluronic acid as a viscous carrier for the microspheres, the hyaluronic acid has a viscosity at 25° C. of between about 140,000 cps and about 500,000 cps at a shear rate of 0.1/second, thereby forming a biocompatible, injectable intraocular drug delivery system which can be injected into an intraocular location through a 20 to 30 gauge syringe needle.

Notably, with our drug delivery system about one hour after the intravitreal injection only about 10% or less, 5% or less or 3% or less of the microspheres are present in the vitreous free of the polymeric matrix.

Our invention also includes a process for making an intraocular pharmaceutical composition by mixing an aqueous suspension of a plurality of corticosteroid particles and an aqueous solution of a viscous polymeric matrix, so that the resulting pharmaceutical composition has a viscosity of between about 130,000 cps and about 300,000 cps at a shear rate of about 0.1/second at about 25° C. The microspheres can have a stable diameter for at least three months after the pharmaceutical has been made and stored for three months in a syringe placed horizontally at about 25° C. at about 60% relative humidity.

Finally, our invention includes a method for treating an articular or spinal pathology by peripheral injecting into a patient of a viscous pharmaceutical composition comprising a plurality of corticosteroid incorporating microspheres mixed into a viscous polymeric matrix, wherein the pharmaceutical composition has a viscosity of between about 130,000 cps and about 300,000 cps at a shear rate of about 0.1/second at about 25° C., such that about one hour after the peripheral injection only about 10% or less of the microspheres are present in vivo free of the polymeric matrix. For epidural injections, especially with transforaminal injections to localize the drug near the spinal root, the optional addition of an anesthetic to the formulation, such as bupivacaine, can be helpful to decrease injection-related discomfort and immediately reduce acute back pain.

The therapeutic agent incorporating microspheres with the therapeutic agent homogenously distributed or dispersed throughout a PLGA or PLA polymeric matrix can be made using known emulsion/solvent evaporation techniques.

Our formulation can comprise an amount of the drug incorporating microspheres sufficient to provide a therapeutic dose of the drug mixed with sodium hyaluronate in an amount which completely envelops the microspheres followed by addition of water and buffers to provide respectively the desired viscosity and an isotonic formulation. Thus, the formulations contain a sufficient concentration of high molecular weight sodium hyaluronate so as to form a gelatinous plug or drug depot upon intravitreal injection into a human eye. The drug containing microspheres are, in effect, trapped or held within this viscous plug, so that undesirable pluming does not occur, and the risk of drug containing microspheres disadvantageously settling directly on the retinal tissue is substantially reduced, for example, relative to using a composition with a water-like viscosity, such as Kenalog® 40. Since sodium hyaluronate solutions are subject to dramatic shear thinning, these formulations are easily injected through 27 gauge or even 30 gauge needles.

Preferably the average molecular weight of the hyaluronate used is less than about 2 million, and more preferably the average molecular weight of the hyaluronate used is between about 1.3 million and 1.6 million. The drug containing microparticles are trapped or held within this viscous plug of hyaluronate, so that undesirable pluming does not occur upon intravitreal injection of the formulation. Thus, the risk of microspheres disadvantageously settling directly on the retinal tissue is substantially reduced, for example, relative to using a composition with a water-like viscosity, such as Kenalog® 40. Since sodium hyaluronate solutions are subject to dramatic shear thinning, these formulations are easily injected through 27 gauge or even 30 gauge needles. The most preferred viscosity range for our formulations is 140,000 cps to 280,000 cps at a shear rate 0.1/second at 25° C.

### DRAWINGS

The following drawings illustrate aspects and features of our invention.

FIG. 1 is a magnetic resonance imaging (MRI) scan of the left eye of a rat into which has been intravitreally injected iron coated microspheres in phosphate buffered saline (PBS).

FIG. 2 consists of three MRI scans of the right eye of the same rat in FIG. 1, which has been intravitreally injected with iron coated microspheres in cross-linked hyaluronic acid, the scans taken at 68 minutes after injection (FIG. 2A), at 155 minutes after injection (FIG. 2B) and at 26 hours and 22 minutes after injection (FIG. 2C).

### DESCRIPTION

Our invention is based upon the discovery of particular drug delivery system formulations and methods for administering these drug delivery systems. The present invention encompasses drug delivery systems which are structured and configured solely for intraocular, as opposed to topical or systemic, administration. The intraocular administration can be by implantation or injection. The drug delivery system within the scope of our invention comprises a plurality of biodegradable microspheres. The therapeutic agent can be released from drug delivery systems made according to the present invention for a period of time between about 3 days to 12 months or more.

The anterior sub-Tenon, anterior suprachoroidal space and anterior intrascleral locations extend from the corneal limbus (the location where the cornea meets the sclera) to approximately 2 mm to 10 mm posteriorly along the surface of the human eye. Further than about 10 mm from the corneal limbus one encounters posterior sub-Tenon, posterior suprachoroidal space and posterior intrascleral locations.

The exterior surface of the globe mammalian eye can have a layer of tissue known as Tenon's capsule, underneath which lies the sclera, followed by the choroid. Between Tenon's capsule and the sclera is a virtual space known as a sub-Tenon space. Another virtual space lies between the sclera and the choroid, referred to as the suprachoroidal space. Delivery of a therapeutic agent to an ocular location the front of the eye (such as the ciliary body) can be facilitated by placement of a suitably configured drug delivery system to a location such as the anterior sub-Tenon space, the anterior suprachoroidal space. Additionally, a drug delivery system can be administered within the sclera, for example to an anterior intrascleral location. Upon lateral movement of the therapeutic agent from such drug delivery implant locations it can diffuse or be transported through the conjunctiva and sclera to the cornea. Upon perpendicular movement of the therapeutic agent through the sclera and/or the choroid it can be delivered to anterior structures of the eye. For example, an aqueous humor suppressant for the treatment of ocular hypertension or glaucoma, can be delivered from drug delivery systems placed in the anterior sub-Tenon space, the suprachoroidal space or intrascleral to the region of the ciliary body.

As can be understood an intrascleral administration of a drug delivery system does not place the drug delivery system as close to the vitreous as does a suprachoroidal (between the sclera and the choroid) administration. For that reason an intrascleral administration of a drug delivery system can be preferred over a suprachoroidal administration so as to reduce the possibility of inadvertently accessing the vitreous upon administration of the drug delivery system.

Additionally, since the lymphatic network resides in or above the tenon's fascia of the eye and deeper ocular tissues have a reduced blood flow velocity, administration of a drug delivery system in a sub-tenon and more eye interior location can provide the dual advantages of avoiding the rapid removal of the therapeutic agent by the ocular lymphatic system (reduced lymphatic drainage) and the presence of only a low circulatory removal of the therapeutic agent from the administration site. Both factors favor passage of effective amounts of the therapeutic agent to the ciliary body and trabecular meshwork target tissue.

In one embodiment of our invention, a drug delivery system for intraocular administration (i.e. by implantation in the sub-Tenon space) comprises configured, consists of, or consists essentially of at least a 75 weight percent of a PLA and no more than about a 25 weight percent of a poly(D,L-lactide-co-glycolide) polymer.

The ciliary body region does not show a rapid rate of drug clearance. Hence we postulate that a therapeutic agent administered by an intraocular administration, such as by a subconjunctival injection, at the equator of the eye can from that location enter the eye to reach the ciliary body region. The anterior sub-Tenon space is location for administration of a drug delivery system because from this location a therapeutic agent released from a drug delivery system can be expected to diffuse to or be transported to the ciliary body region (the target tissue). In other words, administration of a drug delivery system to the anterior sub-Tenon space can efficiently deliver an aqueous humor (elevated IOP) suppressants to the ciliary body region to treat ocular conditions such as ocular hypertension and glaucoma. For the purpose of our invention the anterior sub-Tenon, anterior suprachoroidal space and anterior intrascleral locations can be defined to extend from the corneal limbus (the location where the cornea meets the sclera) to approximately 2 to 10 mm posteriorly along the surface of the human eye. A good location for aqueous humor suppressants entering through this region is the nonpigmented ciliary epithelium where the aqueous humor in produced. Other tissues that would be accessed with a drug delivery system in an anterior intraocular (such as sub-Tenon's) location can be the ciliary body stroma, iris root, and the trabecular meshwork. Therapeutic agents which reduce intraocular pressure primarily by improving uveoscleral flow, such as the prostamides and prostaglandins, would be efficiently delivered with a delivery system in the anterior sub-Tenon's area.

Preferred drug delivery systems are sustained-release microspheres. Drug delivery systems within the scope of our invention can be placed anteriorly in the eye over the ciliary body region with an intrascleral, suprachoroidal, or intravitreal location.

Within the scope of our invention are suspensions of microspheres which can be administered to an intraocular location through a syringe needle. Administration of such a suspension requires that the viscosity of the microsphere suspension at 20° C. be less than about 300,000 to 500,000 cP. The viscosity of water at 20° C. is 1.002 cP (cP is centiposie, a measure of viscosity). The viscosity of olive oil is 84 cP, of castor oil 986 P and of glycerol 1490 cP.

The implants of our invention can include a therapeutic agent mixed with or dispersed within a biodegradable polymer. The implant compositions can vary according to the preferred drug release profile, the particular active agent used, the ocular condition being treated, and the medical history of the patient. Therapeutic agents which can be used in our drug delivery systems include, but are not limited to (either by itself in a drug delivery system within the scope of the present invention or in combination with another therapeutic agent): ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, anti-inflammatory agents, antihypertensives, pressors, antibacterials, antivirals, antifungals, antiprotozoals, anti-infectives, antitumor agents, antimetabolites, antiangiogenic agents, tyrosine kinase inhibitors, antibiotics such as aminoglycosides such as gentamycin, kanamycin, neomycin, and vancomycin; amphenicols such as chloramphenicol; cephalosporins, such as cefazolin HCl; penicillins such as ampicillin, penicillin, carbenicillin, oxycillin, methicillin; lincosamides such as lincomycin; polypeptide antibiotics such as polymixin and bacitracin; tetracyclines such as tetracycline; quinolones such as ciproflaxin, etc.; sulfonamides such as chloramine T; and sulfones such as sulfanilic acid as the hydrophilic entity, anti-viral drugs, e.g. acyclovir, gancyclovir, vidarabine, azidothymidine, azathioprine, dideoxyinosine, dideoxycytosine, dexamethasone, ciproflaxin, water soluble antibiotics, such as acyclovir, gancyclovir, vidarabine, azidothymidine, dideoxyinosine, dideoxycytosine; epinephrine; isoflurphate; adriamycin; bleomycin; mitomycin; ara-C; actinomycin D; scopolamine; and the like, analgesics, such as codeine, morphine, keterolac, naproxen, etc., an anesthetic, e.g. lidocaine; beta.-adrenergic blocker or beta.-adrenergic agonist, e.g. ephidrine, epinephrine, etc.; aldose reductase inhibitor, e.g. epalrestat, ponalrestat, sorbinil, tolrestat; antiallergic, e.g. cromolyn, beclomethasone, dexamethasone, and flunisolide; colchicine, anihelminthic agents, e.g. ivermectin and suramin sodium; antiamebic agents, e.g. chloroquine and chlortetracycline; and antifungal agents, e.g. amphotericin, etc., anti-angiogenesis compounds such as anecortave acetate, retinoids such as Tazarotene, anti-glaucoma agents, such as brimonidine (Alphagan and Alphagan P), acetozolamide, bimatoprost (Lumigan), timolol, mebefunolol; memantine, latanoprost (Xalatan); alpha-2 adrenergic receptor agonists; 2-methoxyestradiol; anti-neoplastics, such as vinblastine, vincristine, interferons; alpha, beta and gamma, antimetabolites, such as folic acid analogs, purine analogs, and pyrimidine analogs; immunosuppressants such as azathiprine, cyclosporine and mizoribine; miotic agents, such as carbachol, mydriatic agents such as atropine, protease inhibitors such as aprotinin, camostat, gabexate, vasodilators such as bradykinin, and various growth factors, such epidermal growth factor, basic fibroblast growth factor, nerve growth factors, carbonic anhydrase inhibitors, and the like.

In one variation the active agent is methotrexate. In another variation, the active agent is a retinoic acid. In another variation, the active agent is an anti-inflammatory agent such as a nonsteroidal anti-inflammatory agent. Nonsteroidal anti-inflammatory agents that may be used include, but are not limited to, aspirin, diclofenac, flurbiprofen, ibuprofen, ketorolac, naproxen, and suprofen. In a further variation, the anti-inflammatory agent is a steroidal anti-inflammatory agent, such as dexamethasone.

Our invention also incompasses a drug delivery device in which the active agent is the active agent is a compound that blocks or reduces the expression of VEGF receptors (VEGFR) or VEGF ligand including but not limited to anti-VEGF aptamers (e.g. Pegaptanib), soluble recombinant decoy receptors (e.g. VEGF Trap), anti-VEGF monoclonal antibodies (e.g. Bevacizamab) and/or antibody fragments (e.g. Ranibizamab), small interfering RNA's decreasing expression of VEGFR or VEGF ligand, post-VEGFR blockade with tyrosine kinase inhibitors, MMP inhibitors, IGFBP3, SDF-1 blockers, PEDF, gamma-secretase, Delta-like ligand 4, integrin antagonists, HIF-1alpha blockade, protein kinase CK2 blockade, and inhibition of stem cell (i.e. endothelial progenitor cell) homing to the site of neovascularization using vascular endothelial cadherin (CD-144) and stromal derived factor (SDF)-1 antibodies.

Steroidal anti-inflammatory agents that can be used in our drug delivery systems can include, but are not limited to, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives.

In one embodiment, cortisone, dexamethasone, fluocinolone, hydrocortisone, methylprednisolone, prednisolone, prednisone, and triamcinolone, and their derivatives, are preferred steroidal anti-inflammatory agents. In another preferred variation, the steroidal anti-inflammatory agent is dexamethasone. In another variation, the biodegradable implant includes a combination of two or more steroidal anti-inflammatory agents.

The active agent, such as a steroidal anti-inflammatory agent, can comprise from about 10% to about 90% by weight of the implant. In one variation, the agent is from about 40% to about 80% by weight of the implant. In a preferred variation, the agent comprises about 60% by weight of the implant. In a more preferred embodiment of the present invention, the agent can comprise about 50% by weight of the implant.

The therapeutic active agent present in our drug delivery systems can be homogeneously dispersed in the biodegradable polymer of the microspheres. The microspheres can be made, for example, by a solvent evaporation method. The selection of the biodegradable polymer used can vary with the desired release kinetics, patient tolerance, the nature of the disease to be treated, and the like. Polymer characteristics that are considered include, but are not limited to, the biocompatibility and biodegradability at the site of implantation, compatibility with the active agent of interest, and processing temperatures. The biodegradable polymer matrix usually comprises at least about 10, at least about 20, at least about 30, at least about 40, at least about 50, at least about 60, at least about 70, at least about 80, or at least about 90 weight percent of the implant. In one variation, the biodegradable polymer matrix comprises about 40% to 50% by weight of the implant.

Biodegradable polymers which can be used include, but are not limited to, polymers made of monomers such as organic esters or ethers, which when degraded result in physiologically acceptable degradation products. Anhydrides, amides, orthoesters, or the like, by themselves or in combination with other monomers, may also be used. The polymers are generally condensation polymers. The polymers can be crosslinked or non-crosslinked. If crosslinked, they are usually not more than lightly crosslinked, and are less than 5% crosslinked, usually less than 1% crosslinked.

For the most part, besides carbon and hydrogen, the polymers will include oxygen and nitrogen, particularly oxygen. The oxygen may be present as oxy, e.g., hydroxy or ether, carbonyl, e.g., non-oxo-carbonyl, such as carboxylic acid ester, and the like. The nitrogen can be present as amide, cyano, and amino. An exemplary list of biodegradable polymers that can be used are described in Heller, Biodegradable Polymers in Controlled Drug Delivery, In: “CRC Critical Reviews in Therapeutic Drug Carrier Systems”, Vol. 1. CRC Press, Boca Raton, Fla. (1987).

Of particular interest are polymers of hydroxyaliphatic carboxylic acids, either homo- or copolymers, and polysaccharides. Included among the polyesters of interest are homo- or copolymers of D-lactic acid, L-lactic acid, racemic lactic acid, glycolic acid, caprolactone, and combinations thereof. Copolymers of glycolic and lactic acid are of particular interest, where the rate of biodegradation is controlled by the ratio of glycolic to lactic acid. The percent of each monomer in poly(lactic-co-glycolic)acid (PLGA) copolymer may be 0-100%, about 15-85%, about 25-75%, or about 35-65%. In certain variations, 25/75 PLGA and/or 50/50 PLGA copolymers are used. In other variations, PLGA copolymers are used in conjunction with polylactide polymers.

Biodegradable polymer matrices that include mixtures of hydrophilic and hydrophobic ended PLGA may also be employed, and are useful in modulating polymer matrix degradation rates. Hydrophobic ended (also referred to as capped or end-capped) PLGA has an ester linkage hydrophobic in nature at the polymer terminus. Typical hydrophobic end groups include, but are not limited to alkyl esters and aromatic esters. Hydrophilic ended (also referred to as uncapped) PLGA has an end group hydrophilic in nature at the polymer terminus. PLGA with a hydrophilic end groups at the polymer terminus degrades faster than hydrophobic ended PLGA because it takes up water and undergoes hydrolysis at a faster rate. Examples of suitable hydrophilic end groups that may be incorporated to enhance hydrolysis include, but are not limited to, carboxyl, hydroxyl, and polyethylene glycol. The specific end group will typically result from the initiator employed in the polymerization process. For example, if the initiator is water or carboxylic acid, the resulting end groups will be carboxyl and hydroxyl. Similarly, if the initiator is a monofunctional alcohol, the resulting end groups will be ester or hydroxyl.

Other agents may be employed in a drug delivery system formulation for a variety of purposes. For example, buffering agents and preservatives may be employed. Preservatives which may be used include, but are not limited to, sodium bisulfite, sodium bisulfate, sodium thiosulfate, benzalkonium chloride, chlorobutanol, thimerosal, phenylmercuric acetate, phenylmercuric nitrate, methylparaben, polyvinyl alcohol and phenylethyl alcohol. Examples of buffering agents that may be employed include, but are not limited to, sodium carbonate, sodium borate, sodium phosphate, sodium acetate, sodium bicarbonate, and the like, as approved by the FDA for the desired route of administration. Electrolytes such as sodium chloride and potassium chloride may also be included in the formulation.

The biodegradable drug delivery systems can also include additional hydrophilic or hydrophobic compounds that accelerate or retard release of the active agent. Additionally, release modulators such as those described in U.S. Pat. No. 5,869,079 can be included in the implants. The amount of release modulator employed will be dependent on the desired release profile, the activity of the modulator, and on the release profile of the glucocorticoid in the absence of modulator. Where the buffering agent or release enhancer or modulator is hydrophilic, it may also act as a release accelerator. Hydrophilic additives act to increase the release rates through faster dissolution of the material surrounding the drug particles, which increases the surface area of the drug exposed, thereby increasing the rate of drug diffusion. Similarly, a hydrophobic buffering agent or enhancer or modulator can dissolve more slowly, slowing the exposure of drug particles, and thereby slowing the rate of drug diffusion.

A drug delivery system within the scope of the present invention can be formulated with particles of an active agent dispersed within a biodegradable polymer matrix of microspheres. Without being bound by theory, it is believed that the release of the active agent can be achieved by erosion of the biodegradable polymer matrix and by diffusion of the particulate agent into an ocular fluid, e.g., the vitreous, with subsequent dissolution of the polymer matrix and release of the active agent. Factors which influence the release kinetics of active agent from the implant can include such characteristics as the size and shape of the implant, the size of the active agent particles, the solubility of the active agent, the ratio of active agent to polymer(s), the method of manufacture, the surface area exposed, and the erosion rate of the polymer(s). The release kinetics achieved by this form of active agent release are different than that achieved through formulations which release active agents through polymer swelling, such as with crosslinked hydrogels. In that case, the active agent is not released through polymer erosion, but through polymer swelling and drug diffusion, which releases agent as liquid diffuses through the pathways exposed.

The release rate of the active agent can depend at least in part on the rate of degradation of the polymer backbone component or components making up the biodegradable polymer matrix. For example, condensation polymers may be degraded by hydrolysis (among other mechanisms) and therefore any change in the composition of the implant that enhances water uptake by the implant will likely increase the rate of hydrolysis, thereby increasing the rate of polymer degradation and erosion, and thus increasing the rate of active agent release.

The release kinetics of the implants of the present invention can be dependent in part on exposed the surface area of the microspheres. A larger surface area exposes more polymer and active agent to ocular fluid, causing faster erosion of the polymer matrix and dissolution of the active agent particles in the fluid. Therefore, the size and shape of the implant may also be used to control the rate of release, period of treatment, and active agent concentration at the site of implantation. At equal active agent loads, larger implants will deliver a proportionately larger dose, but depending on the surface to mass ratio, may possess a slower release rate. For implantation in an ocular region, the total weight of the population of therapeutic agent incorporating microspheres injected preferably ranges, e.g., from about 100 μg to about 15 mg. More preferably, from about 300 μg to about 10 mg, and most preferably from about 500 μg to about 5 mg. In a particularly preferred embodiment of the present invention the weight of an implant is between about 500 μg and about 2 mg, such as between about 500 μg and about 1 mg. The bioerodible implants are microspheres with diameter between about 1 micron and about 150 microns.

Examples of fast release microspheres include those made of certain lower molecular weight, fast degradation profile polylactide polymers, such as R104 made by Boehringer Ingelheim GmbH, Germany, which is a poly(D,L-lactide) with a molecular weight of about 3,500. Examples of medium release rate microspheres include those made of certain medium molecular weight, intermediate degradation profile PLGA copolymers, such as RG755 made by Boehringer Ingelheim GmbH, Germany, which is a poly(D,L-lactide-co-glycolide with wt/wt 75% lactide:25% glycolide, a molecular weight of about 40,000 and an inherent viscosity of 0.50 to 0.70 dl/g. Examples of slow release microspheres include those made of certain other high molecular weight, slower degradation profile polylactide polymers, such as R203/RG755 made by Boehringer Ingelheim GmbH, Germany, for which the molecular weight is about 14,000 for R203 (inherent viscosity of 0.25 to 0.35 dl/g) and about 40,000 for RG755. When administered together, these microspheres provide for an extend continuous release of drug over a period of at least 120 days in vitro which can result in sustained drug levels (concentration) of at least about 5-10 ng dexamethasone equivalent/mL in the vitreous (i.e. in vivo) for up to about 240 days.

In general, the present intraocular drug delivery systems comprise a therapeutic agent (such as a corticosteroid) incorporated within biocompatible, biodegradable microspheres and a viscous carrier for the microspheres. One of the important advantages of the present drug delivery system is that is compatible with or friendly to the tissues in the posterior segment of the eye.

The therapeutic agent present in the microspheres is present in the drug delivery system in a therapeutically effective amount, that is in an amount effective in providing a desired therapeutic effect in the eye into which the drug delivery system is placed. The viscous carrier is present in an effective amount in increasing, advantageously substantially increasing, the viscosity of the drug delivery system. Without wishing to limit the invention to any particular theory of operation, it is believed that increasing the viscosity of the drug delivery system to values well in excess of the viscosity of water, for example, at least about 100 cps at a shear rate of 0.1/second at 25° C., are highly effective for placement, e.g., injection, into the posterior segment of an eye of a human or animal are obtained. Along with the advantageous placement or injectability of the present drug delivery systems into the posterior segment, the relatively high viscosity of the present drug delivery systems are believed to enhance the ability of the present drug delivery systems to maintain the microspheres in substantially uniform suspension in the drug delivery systems for prolonged periods of time, for example, for at least about one week, without requiring resuspension processing.

Advantageously, the viscous carrier and therefore the drug delivery system has a viscosity at 25° C. of at least about 10 cps or at least about 100 cps or at least about 1000 cps, more preferably at least about 10,000 cps and still more preferably at least about 70,000 cps or more, for example up to about 200,000 cps or about 250,000 cps, or about 300,000 cps or more, at a shear rate of 0.1/second. The present drug delivery systems not only have the relatively high viscosity as noted above but also have the ability or are structured or made up so as to be effectively placeable, e.g., injectable, into a posterior segment of an eye of a human or animal, preferably through a 27 gauge needle, or even through a 30 gauge needle.

The presently useful viscous carrier preferably is a shear thinning component in that as the present composition containing such a shear thinning viscous carrier is passed or injected into the posterior segment of an eye, for example, through a narrow space, such as 27 gauge needle, under high shear conditions the viscosity of the viscous carrier is substantially reduced during such passage. After such passage, the viscous carrier regains substantially its preinjection viscosity so as to maintain the microspheres in suspension in the eye.

Any suitable viscous carrier, for example, ophthalmically acceptable viscous carrier, may be employed in accordance with the present invention. The viscous carrier is present in an amount effective in providing the desired viscosity to the drug delivery system. Advantageously, the viscous carrier is present in an amount in a range of from about 0.5 wt % to about 95 wt % of the drug delivery system. Specific amount of the viscous carrier used depends upon a number of factors including, for example and without limitation, the specific viscous carrier used, the molecular weight of the viscous carrier used, the viscosity desired for the present drug delivery system being produced and/or used and like factors.

Examples of useful viscous carriers include, but are not limited to, hyaluronic acid, carbomers, polyacrylic acid, cellulosic derivatives, polycarbophil, polyvinylpyrrolidone, gelatin, dextrin, polysaccharides, polyacrylamide, polyvinyl alcohol, polyvinyl acetate, derivatives thereof and mixtures thereof.

A dermal filler can also be used as the viscous carrier. Suitable dermal fillers for that purpose include collagen (sterile collagen is sold under the trade names Zyderm, Zyplast, Cosmoderm, Cosmoplast and Autologen), Hylaform® (hyaluronic acid), Restylane® (hyaluronic acid), Sculptra™ (polylactic acid), Radiesse™ (calcium hydoxyl apatite) and Juvederm™. Juvederm™ available from Allergan, Inc. (Irvine, Calif.) comprises a sterile, biodegradable, non-pyrogenic, viscoelastic, clear, colorless, homogenized gel consisting of cross-linked hyaluronic acid formulated at a concentration of 24 gm/ml in a physiologic buffer. Hyaluronic acid is a polysaccharide found in the dermis of all mammals.

The molecular weight of the presently useful viscous carrier can be in a range of about 10,000 Daltons or less to about 2 million Daltons or more. In one particularly useful embodiment, the molecular weight of the viscous carrier is in a range of about 100,000 Daltons or about 200,000 Daltons to about 1 million Daltons or about 1.5 million Daltons. Again, the molecular weight of the viscous carrier useful in accordance with the present invention, may vary over a substantial range based on the type of viscous carrier employed, and the desired final viscosity of the present drug delivery system in question, as well as, possibly one or more other factors.

In one very useful embodiment, the viscous carrier is a polymeric hyaluronate component, for example, a metal hyaluronate component, preferably selected from alkali metal hyaluronates, alkaline earth metal hyaluronates and mixtures thereof, and still more preferably selected from sodium hyaluronates, and mixtures thereof. The molecular weight of such hyaluronate component preferably is in a range of about 50,000 Daltons or about 100,000 Daltons to about 1.3 million Daltons or about 2 million Daltons. In one embodiment, the present compositions include a polymeric hyaluronate component in an amount in a range about 0.05% to about 0.5% (w/v). In a further useful embodiment, the hyaluronate component is present in an amount in a range of about 1% to about 4% (w/v) of the composition. In this latter case, the very high polymer viscosity forms a gel that slows particle sedimentation rate to the extent that often no resuspension processing is necessary over the estimated shelf life, for example, at least about 2 years, of the drug delivery system. Such a drug delivery system can be marketed in pre-filled syringes since the gel cannot be easily removed by a needle and syringe from a bulk container.

The drug delivery can also include an aqueous carrier component which is advantageously ophthalmically acceptable and may include one or more conventional excipients useful in ophthalmic compositions.

The present drug delivery system can include at least one buffer component in an amount effective to control the pH of the drug delivery system and/or at least one tonicity component in an amount effective to control the tonicity or osmolality of the drug delivery system. More preferably, the present drug delivery systems include both a buffer component and a tonicity component. The buffer component and tonicity component may be chosen from those which are conventional and well known in the ophthalmic art.

Examples of such buffer components include, but are not limited to, acetate buffers, citrate buffers, phosphate buffers, borate buffers and the like and mixtures thereof. Phosphate buffers are particularly useful. Useful tonicity components include, but are not limited to, salts, particularly sodium chloride, potassium chloride, any other suitable ophthalmically acceptably tonicity component and mixtures thereof.

The amount of buffer component employed preferably is sufficient to maintain the pH of the drug delivery system in a range of about 6 to about 8, more preferably about 7 to about 7.5. The amount of tonicity component employed preferably is sufficient to provide an osmolality to the present drug delivery system in a range of about 200 to about 400, more preferably about 250 to about 350, mOsmol/kg respectively. Advantageously, the present drug delivery systems are substantially isotonic.

Methods of using the present drug delivery are provided and are included within the scope of the present invention. In general, such methods comprise administering a drug delivery system in accordance with the present invention to a posterior segment of an eye of a human or animal, thereby obtaining a desired therapeutic effect. The administering step advantageously comprises at least one of intravitreal injecting, subconjunctival injecting, sub-tenon injecting, retrobulbar injecting, suprachoroidal injecting and the like. A syringe apparatus including an appropriately sized needle, for example, a 27 gauge needle or a 30 gauge needle, can be effectively used to inject the drug delivery system with the posterior segment of an eye of a human or animal.

The drug delivery systems disclosed herein can be used to prevent or to treat various ocular diseases or conditions, including the following: maculopathies/retinal degeneration: macular degeneration, including age related macular degeneration (ARMD), such as non-exudative age related macular degeneration and exudative age related macular degeneration, choroidal neovascularization, retinopathy, including diabetic retinopathy, acute and chronic macular neuroretinopathy, central serous chorioretinopathy, and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease. Traumatic/surgical: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors. Miscellaneous: punctate inner choroidopathy, acute posterior multifocal placoid pigment epitheliopathy, myopic retinal degeneration, acute retinal pigment epithelitis and the like.

### EXAMPLES

The following examples illustrate aspects and embodiments of our invention.

### Example 1

### Sustained Release Microsphere Hyaluronic Acid Formulation

In this experiment we made and injected in vivo a particular new microsphere formulation. This formulation was developed to address several problems with existing intraocular microsphere formulations. Thus, due to the large amount of exposed surface area biodegradable, drug-incorporating microspheres injected into an intraocular location such an intra-scleral or intravitreal location of a mammalian eye rapidly (that is typically within minutes to a few hours, depending upon the biodegradable polymer used to make the microspheres) release all or most incorporated drug. Thus, a 50/50 blend (50% glycolide monomers in the PLGA polymeric matrix and 50% lactide monomers in the PLGA polymeric matrix) will release about 100% of incorporated drug over a 1 hour to about 24 hour period, depending upon the particular acid end/ester end mix of the PLGA polymers used. Such rapid drug release necessitates frequent re-dosing with the microspheres to provide a therapeutic effect without provision of a toxic amount of the drug level. Additionally, as a foreign element injected microspheres can be immunogenic.

Significantly, we determined that intraocular injection of drug-incorporating, biodegradable microspheres formulated in a viscous carrier permits a substantially linear (first order) release of the drug over a 1 to 60 day period to be achieved, the time period of linear drug release depending primarily upon (when other factors such as the PLGA used to make the microspheres is held constant) the degree of cross linking of the particular viscous carrier.

It should be noted that the intraocular (in vivo) release kinetics of a drug from drug incorporating microspheres in a viscous carrier, is very different from the intraocular release kinetics of the same drug from the same viscous carrier (i.e. in the absence of microspheres). This is because in the later case the release kinetics are determined primarily by the rate of solubilization of the drug into the aqueous intraocular medium, while in the former case the release kinetics are much more complex as they are determined by a combination of the rate of release of the drug from the microspheres (which in turn depends eg upon the rate at which the microspheres become hydrated, the rate at which the microspheres biodegrade/bioerode, the rates at which drug is released from the surface, from near the surface and from the interior of the microspheres), the rate of hydration of the viscous carrier, and the rate of degradation of the viscous carrier, the later two factors being related to the degree of cross-linking of the viscous carrier.

Thus, we developed a hyaluronic acid (HA) formulation which can permit both a sustained release of therapeutic agent from biodegradable microspheres and as well reduce inflammatory potential of the injected microspheres. HA is a substance which is native to the vitreous humor and inherently has anti-inflammatory properties through inhibition of inflammatory mediators (cytokines and prostaglandins), inhibition of macrophage motility, scavenging oxygen free radicals, and inhibition of matrix metalloproteinases. Liao Y., et al., Hyaluronan pharmaceutical characterization and drug delivery, Drug Deliv; 12: 327-42.

The microsphere-HA formulation we developed provides a sustained release drug depot. The therapeutic agent is released from the drug depot and the release kinetics are determined by at least four separate rate limiting factors: (1) the rate of water ingress into the HA from the surrounding vitreous, which rate decreases as the degree of cross-linking of the HA is increased; (2) the rate of and extent to which the biodegradable polymer(s) constituting the microspheres hydrates, swell and release drug from the microspheres; (3) the rate at which microsphere released drug diffuses through the surrounding HA polymers and is releases from the gel depot into the vitreous, and; (4) the rate of diffusion or transport of released drug through the vitreous to the target tissue (i.e. the retina). Importantly, the HA gel used in the formulation serves as both as a drug release agent and to hinder microsphere recognition by macrophages and phagocytosis.

To demonstrate that HA can be combined with microspheres to enhance microsphere agglomeration in such a drug depot formulation, the vitreous cavity of a rat was injected with a microsphere-HA formulation. The microspheres were obtained from FerroTrack (Biopal, Worcester, Mass.) as non-bioerodible, 1 micron diameter polystyrene beads coated with iron, which can be imaged by MRI. The concentration of microspheres used in the stock solution was 8.4×10⁻⁹ particles per milliliter. 23.89 ml of a partially crosslinked HA (Juvederm™) was mixed with 3.14 ml of the microsphere solution resulting in 1.055×10⁻⁹ microsphere particles per ml of the HA hydrogel gel. As a control, an analogous microsphere formulation was made substituting PBS for the HA.

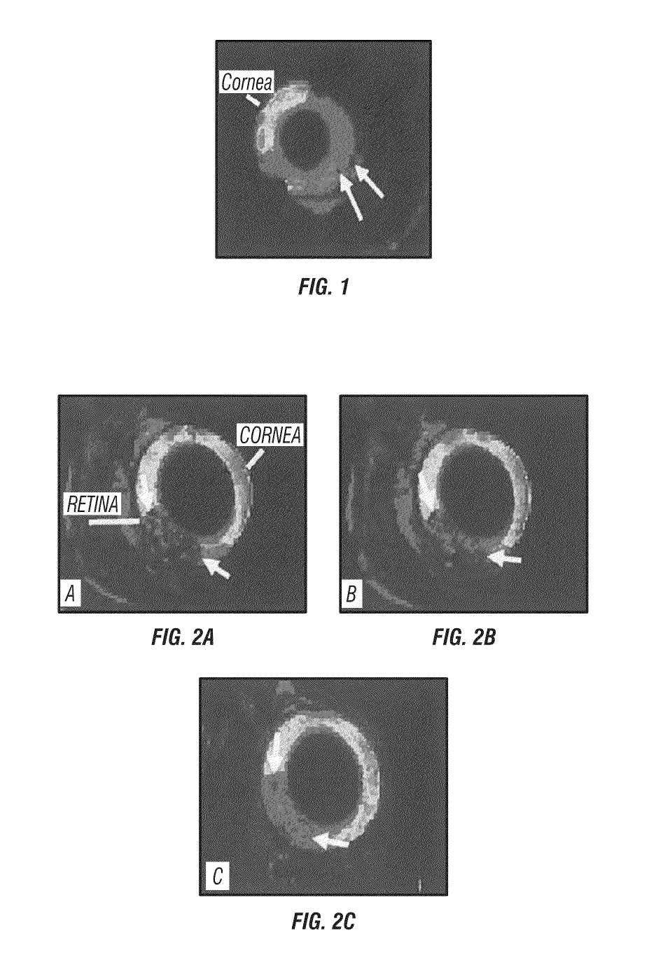

Using a 50 μl Hamilton syringe with a 30G needle, an intravitreal injection of 5 μl was delivered superotemporally 1 mm posterior to the limbus through the pars plana in an anesthetized rat. The microsphere-HA formulation was injected into the right eye. The same microspheres in PBS was injected into the left eye. Using a 7 Tesla high resolution Bruker PharmaScan MRI, serial scans in vivo were performed over a period of about 26 hours. The first MRI scan at 68 minutes after injection showed that the microspheres in PBS were present as a diffuse distribution in the vitreous cavity with about less than 5% of the total injection forming a defined depot (FIG. 1). In contrast at +68 minutes the injected microsphere-HA formulation was present as a distinct depot in the vitreous cavity (FIG. 2A). Over the next 25 hours, the right eye microspheres-HA depot formulation was maintained with excellent consolidation of the microspheres. Water started to diffuse into the microsphere-HA depot by 155 minutes (FIG. 2B), and this continued through the 26 hour time point (FIG. 2C). The animal was rescanned at 95 hours and the appearance of the microsphere-HA depot was the same as it was at the 26 hour time point. Significantly, a final MRI scan was performed at 336 hours and the microsphere-HA depot was still present at the same location.

FIG. 1 is an MRI scan of the left rat eye showing that microspheres in PBS demonstrated poor depot formation with less than about 5% of the injected volume forming a distinct depot (arrows).

FIG. 2 is an MRI scan of the right rat eye following injection with the microsphere-HA formulation. FIG. 2A was taken at +68 minutes and shows at the arrows the microsphere-HA depot formulation in the vitreous cavity; FIG. 2B was taken +155 minute time point and shows visible depot hydration, as the yellow/green areas at the arrows, and; FIG. 2C was taken at +26 hours and 22 minutes and shows that the microsphere-HA formulation is still well defined (arrows) and that progressive hydration of the HA is apparent within the depot.

In conclusion, this study demonstrates that a simple formulation of microspheres in PBS showed poor depot formation in the vitreous cavity. However, a HA/microsphere combination demonstrates excellent depot formation and consolidation of the microspheres with time.

Advantages of our microsphere-HA formulations include:
1. extended or sustained release of therapeutic agent in vivo thereby reducing or eliminating the need to re-dose to effectively treat an ocular condition.
2. reduced immunogenicity of drug-incorporating microspheres in vivo even though present in vivo for a period of days, weeks or months.
3. Reduced visual obscuration due to reduced microsphere dispersion from the drug depot formulation, thereby to improving quality of life for patients.
4. Rapid microsphere agglomeration in viva can increase the in vivo half-life of the drug depot.
5. Encapsulating an active therapeutic agent within biodegradable polymers in the microspheres can thereby protect a therapeutic agent which is a labile protein or peptides in the drug depot.
6. Allows use of prefilled syringes filled with a suspending of microspheres in the HA gel matrix
7. Reduces the potential for hypodermic needle occlusion (needle block) due to lower incidence of microsphere clumping during the injection because of the enhanced lubrication with and suspension of the microspheres by the HA.

In this experiment the microspheres were mixed with a HA. But since HA is a polymeric polyanionic polysaccharide, a positive charge can be applied to the microspheres to create an electrostatic bond with the surrounding HA polymers. This can result is a more reduced rate of therapeutic drug release from the microspheres. Additionally, variations in the HA concentration, molecular weight, and degree of cross-linking can be carried out to thereby control microsphere agglomeration and to enhance microspheres containment within the high viscosity carrier (drug depot).

### Example 2

### Treatment of Macular Edema with Intravitreal Microsphere Suspension

A 64 year old obese female patient with symptoms of diabetes presents with vision loss due to macula edema with central retinal vein occlusion and/or branch retinal vein occlusion. She receives intravitreal injection of a high viscosity formulation comprising triamcinolone acetonide (about 4 mg) incorporating PLGA microspheres (about 10 microns in diameter, 20% drug load) in a high molecular weight (about 900,000 Daltons) polymeric hyaluronate.

Six months after injection she demonstrates an improved best corrected visual acuity of five or more letters from baseline as determined using the Early Treatment of Diabetic Retinopathy Study (ETDRS) visual acuity chart.

All references, articles, patents, applications and publications set forth above are incorporated herein by reference in their entireties.

Accordingly, the spirit and scope of the following claims should not be limited to the descriptions of the preferred embodiments set forth above.

We claim:

1. A biocompatible, injectable intraocular drug delivery system comprising:
(a) a plurality of biodegradable microspheres;
(b) a therapeutic agent incorporated by the microspheres; and
(c) a viscous carrier for the microspheres, the viscous carrier having a viscosity at 25° C. of between about 140,000 cps and about 500,000 cps at a shear rate of 0.1/second, thereby forming a biocompatible, injectable intraocular drug delivery system, wherein the viscous carrier is a partially crosslinked hyaluronic acid, and wherein the microspheres comprise a poly(lactide-co-glycolide) (PLGA) polymer, the drug delivery system such that about one hour after intravitreal injection of the drug delivery system about 5% or less of the microspheres are present in the vitreous free of the viscous carrier.

2. The drug delivery system of claim 1, further comprising an aqueous vehicle for the microspheres.

3. The drug delivery system of claim 1, wherein the therapeutic agent has a solubility in water at 25° C. of between about 0.1 μg/ml and about 1 gm/mi.

4. The drug delivery system of claim 1, wherein the drug delivery system can be injected into an intraocular location through a 25 to 32 gauge syringe needle.

5. The drug delivery system of claim 1 wherein the microspheres are substantially uniformly suspended in the viscous carrier composition.

6. The drug delivery system of claim 1 wherein the therapeutic agent is a corticosteroid.

7. The drug delivery system of claim 1, wherein the microspheres have an average diameter between about 1 micron and about 100 microns.

8. The drug delivery system of claim 1, wherein the therapeutic agent is an anti-VEGF monoclonal antibody.
